(19) United States
(12) Patent Application Publication
(10) Pub. No.: US 2026/0008989 A1
(43) Pub. Date: Jan. 8, 2026

(54) MULTI-SEQUENCE INTEGRITY-PRESERVING MONITORING APPARATUS AND METHOD FOR DEEP-SEA MICROORGANISM CULTIVATION

(71) Applicants: GUANGDONG LABORATORY OF SOUTHERN OCEAN SCIENCE AND ENGINEERING (GUANGZHOU), Guangdong (CN); GUANGDONG UNIVERSITY OF TECHNOLOGY, Guangdong (CN)

(72) Inventors: Jingchun FENG, Guangdong (CN); Si ZHANG, Guangdong (CN); Yuhang ZHANG, Guangdong (CN); Yan XIE, Guangdong (CN)

(73) Assignees: GUANGDONG LABORATORY OF SOUTHERN OCEAN SCIENCE AND ENGINEERING (GUANGZHOU), Guangdong (CN); GUANGDONG UNIVERSITY OF TECHNOLOGY, Guangdong (CN)

(21) Appl. No.: 19/260,286

(22) Filed: Jul. 4, 2025

(30) Foreign Application Priority Data
Jul. 5, 2024 (CN) 202410896784.4

(51) Int. Cl. C12M 1/34; C12M 1/00

(52) U.S. Cl. CPC C12M 41/40; C12M 23/38; C12M 29/20; C12M 41/12; C12M 41/26; C12M 41/32; C12M 41/44

(57) ABSTRACT

The present invention discloses a multi-sequence integrity-preserving monitoring apparatus and method for deep-sea microorganism cultivation, relating to the technical field of marine microorganisms. The apparatus includes a gas pressurization unit, a pressure-retaining cultivation unit, an integrity-preserving monitoring unit, and a sampling control unit. The gas pressurization unit is configured to provide high-pressure nitrogen gas to the pressure-retaining cultivation unit and the integrity-preserving monitoring unit, achieving a high-pressure cultivation environment for deep-sea cultured samples, and change the pressure values of the nitrogen gas injected into the pressure-retaining cultivation cylinder and the pressure-retaining buffer cylinder, to form a pressure difference, thus driving pistons for pressure-retaining transfer of deep-sea cultured samples. The opening and closing of the second multi-way valve enable switching and sampling operations of multi-sequence pressure-retaining cultivation cylinders, and the single integrity-preserving monitoring unit is used to meet the demand for in-situ pressure-retaining sampling and monitoring of multiple pressure-retaining cultivation cylinders.

### CROSS REFERENCE TO THE RELATED APPLICATIONS

This application claims the priority benefit of China application serial no. 202410896784.4, filed on Jul. 5, 2024. The entirety of the above-mentioned patent applications is hereby incorporated by reference herein and made a part of this specification.

### BACKGROUND

### Technical Field

The present invention relates to the technical field of marine microorganisms, and more specifically, to a multi-sequence integrity-preserving monitoring apparatus and method for deep-sea microorganism cultivation.

### Description of Related Art

The deep sea harbors abundant microbial resources, and their unique biochemical processes and metabolites are key to exploring biogeochemical cycles in extreme environments and even the origin of life. High pressure is an important environmental indicator distinguishing the deep sea from other ecosystems, making deep-sea microorganisms generally pressure-tolerant. Therefore, deep-sea microorganisms are closely related to pressure. After removal from a high-pressure environment, their growth and metabolic capabilities may be severely affected, leading to dormancy or even cell rupture and death, greatly limiting the research and application of deep-sea microorganisms. Current research on deep-sea microorganism cultivation primarily maintains microbial growth and metabolic activity through pressure-retaining cultivation, but during sample monitoring, microbial cultured samples still need to be depressurized and sampled for monitoring. Due to the sudden pressure change from depressurization, monitoring microbial cultured samples under normal pressure conditions causes phenomena such as microbial cell rupture, resulting in sample distortion. Meanwhile, since critical stages of the microbial cultivation process require high-frequency monitoring, continuous depressurized sampling causes significant losses in the total volume and nutrients of the cultivation system, which is equally non-negligible for the cultivation system.

### SUMMARY

To overcome the shortcomings of the prior art in deep-sea microorganism cultivation monitoring, which requires depressurized sampling leading to sample distortion and significant sampling losses, the present invention provides a multi-sequence integrity-preserving monitoring apparatus and method for deep-sea microorganism cultivation, enabling low-loss transfer and integrity-preserving monitoring of multi-sequence cultivation systems under full-process pressure retention, meeting the requirements for high-integrity-preserving, low-loss, and multi-parameter monitoring of cultured samples, and extending the pressure-retaining cultivation cycle of samples.

To address the above technical problems, the technical solution of the present invention is as follows:

The present invention provides a multi-sequence integrity-preserving monitoring apparatus for deep-sea microorganism cultivation, including a gas pressurization unit, a pressure-retaining cultivation unit, an integrity-preserving monitoring unit, and a sampling control unit.

The pressure-retaining cultivation unit includes a first multi-way valve, a plurality of pressure-retaining cultivation cylinders, and a second multi-way valve, where an input end of the first multi-way valve is connected to a first output end of the gas pressurization unit, a plurality of output ends of the first multi-way valve are each connected to one end of one pressure-retaining cultivation cylinder correspondingly, and the other end of each of the pressure-retaining cultivation cylinders is connected to a one end of the second multi-way valve correspondingly.

The integrity-preserving monitoring unit includes a three-way valve, a four-way valve, a pressure-retaining buffer cylinder, and a monitoring compartment, where a first port of the four-way valve is connected to an output end of the second multi-way valve, and a second port of the four-way valve is connected to an input end of the monitoring compartment; a third port of the four-way valve is connected to one end of the pressure-retaining buffer cylinder, and the other end of the pressure-retaining buffer cylinder is connected to a first port of the three-way valve; and a fourth port of the four-way valve is connected to a second port of the three-way valve, and a third port of the three-way valve is connected to a second output end of the gas pressurization unit.

An input end of the sampling control unit is connected to a data output end of the monitoring compartment, and an output end of the sampling control unit is connected to a control end of the gas pressurization unit, a control end of the first multi-way valve, a control end of the second multi-way valve, a control end of the three-way valve, and a control end of the four-way valve.

The gas pressurization unit is configured to provide high-pressure nitrogen gas to the pressure-retaining cultivation unit and the integrity-preserving monitoring unit, achieving a high-pressure cultivation environment for deep-sea cultured samples. The gas pressurization unit achieves transfer of deep-sea cultured samples between the pressure-retaining cultivation cylinder, the pressure-retaining buffer cylinder, and the monitoring compartment by changing the pressure values of nitrogen gas injected into the pressure-retaining cultivation cylinder and the pressure-retaining buffer cylinder. The opening and closing of the second multi-way valve enable switching and sampling operations of multi-sequence pressure-retaining cultivation cylinders, and the single integrity-preserving monitoring unit is used to meet the demand for sampling and monitoring of multiple pressure-retaining cultivation cylinders, reducing equipment costs. The opening and closing of the three-way valve and the four-way valve achieve full-process pressure-retaining operations for sampling and recovery of cultured samples, ensuring the authenticity of cultured samples during monitoring, reducing losses of cultured samples during monitoring, improving the sampling frequency and efficiency of the cultivation process, and extending the cultivation cycle of deep-sea microorganisms.

Preferably, each of the pressure-retaining cultivation cylinders includes a gas sealing end cap, a first cavity, a first piston, and a seawater sealing end cap.

The first piston is disposed inside the first cavity and divides the first cavity into a first gas pressurization chamber and a pressure-retaining cultivation chamber; and the gas sealing end cap is disposed at one end of the first gas pressurization chamber, and the seawater sealing end cap is disposed at one end of the pressure-retaining cultivation chamber.

The plurality of output ends of the first multi-way valve are each connected to the gas sealing end cap of one pressure-retaining cultivation cylinder correspondingly, and the seawater sealing end cap of each of the pressure-retaining cultivation cylinders is connected to one input end of the second multi-way valve correspondingly.

The first cavity is a titanium alloy cylindrical structure with a built-in movable first piston, and the first piston is fitted with an O-ring seal, separating the first cavity into the first gas pressurization chamber and the pressure-retaining cultivation chamber. The gas pressurization unit injects high-pressure nitrogen gas into the first gas pressurization chamber of the pressure-retaining cultivation cylinder via the first multi-way valve, achieving a high-pressure cultivation environment for deep-sea cultured samples.

Preferably, the pressure-retaining buffer cylinder includes a first sealing end cap, a second cavity, a second piston, and a second sealing end cap.

The second piston is disposed inside the second cavity and divides the second cavity into a second gas pressurization chamber and a pressure-retaining buffer chamber; and the first sealing end cap is disposed at one end of the pressure-retaining buffer chamber, and the second sealing end cap is disposed at one end of the second gas pressurization chamber.

The third port of the four-way valve is connected to the first sealing end cap of the pressure-retaining buffer cylinder, and the second sealing end cap of the pressure-retaining buffer cylinder is connected to the first port of the three-way valve.

The second cavity is a titanium alloy cylindrical structure with a built-in movable second piston, and the second piston is fitted with an O-ring seal, dividing the second cavity into the second gas pressurization chamber and the pressure-retaining buffer chamber. The gas pressurization unit injects high-pressure nitrogen gas into the second gas pressurization chamber of the pressure-retaining buffer cylinder via the three-way valve, achieving a high-pressure cultivation environment for deep-sea cultured samples. The cultured sample in the pressure-retaining cultivation cylinder enters the pressure-retaining buffer chamber of the pressure-retaining buffer cylinder via the second multi-way valve and the four-way valve, achieving pressure-retaining buffering of the cultured sample.

Preferably, the monitoring compartment includes a third sealing end cap, a fourth sealing end cap, a compartment body, a viewing port, a Raman spectroscopy probe, an ultraviolet spectroscopy probe, a compartment pressure sensor, a water quality sensor, and a liquid level sensor.

The third sealing end cap and the fourth sealing end cap are respectively disposed at two ends of the compartment body, and the viewing port is disposed on a sidewall of the compartment body; and the liquid level sensor is disposed on a lower surface of the third sealing end cap, the water quality sensor and the compartment pressure sensor are disposed on an upper surface of the fourth sealing end cap, and the Raman spectroscopy probe and the ultraviolet spectroscopy probe are all disposed on the viewing port.

The second port of the four-way valve is connected to a lower surface of the fourth sealing end cap of the monitoring compartment.

Data output ends of the Raman spectroscopy probe, the ultraviolet spectroscopy probe, the water quality sensor, and the liquid level sensor are all connected to the input end of the sampling control unit.

The compartment body is a titanium alloy cylindrical structure, and the fourth sealing end cap is communicated with the four-way valve for injection and outflow of cultured samples. The compartment pressure sensor and the liquid level sensor are configured to monitor the pressure value and liquid level height of the cultured sample in the compartment body in real time, reflecting the sample volume in the compartment body. The Raman spectroscopy probe, the ultraviolet spectroscopy probe, and the water quality sensor monitor multiple physicochemical parameters of the cultured sample in the compartment body in real time. The gas pressurization unit injects high-pressure nitrogen gas into the compartment body via the three-way valve and the four-way valve, achieving a high-pressure monitoring environment for deep-sea cultured samples.

Preferably, the water quality sensor includes any one or more of a methane sensor, a carbon dioxide sensor, a dissolved oxygen sensor, a temperature sensor, a conductivity sensor, and a pH sensor.

The methane sensor, the carbon dioxide sensor, the dissolved oxygen sensor, the temperature sensor, the conductivity sensor, and the pH sensor are respectively configured to detect the methane concentration, the carbon dioxide concentration, the dissolved oxygen concentration, the temperature, the conductivity, and the pH value of the cultured sample.

Preferably, the dissolved oxygen sensor, the temperature sensor, the conductivity sensor, and the pH sensor are all probe-type sensors.

Probe-type sensors can reduce the area occupied on the fourth sealing end cap, facilitating arrangement.

Preferably, the apparatus further includes a plurality of sampling needle valves and a plurality of ventilation needle valves.

One ventilation needle valve is disposed between each of the output ends of the first multi-way valve and one end of one pressure-retaining cultivation cylinder.

One ventilation needle valve is disposed between the other end of the pressure-retaining buffer cylinder and the first port of the three-way valve.

One sampling needle valve is disposed between the other end of each of the pressure-retaining cultivation cylinders and one input end of the second multi-way valve.

One sampling needle valve is disposed between the third port of the four-way valve and one end of the pressure-retaining buffer cylinder.

One sampling needle valve is disposed between the second port of the four-way valve and the input end of the monitoring compartment.

Preferably, the apparatus further includes a plurality of pressure sensors.

Each of the pressure sensors is disposed at a sampling end of one sampling needle valve correspondingly, and a data output end of each of the pressure sensors is connected to the input end of the sampling control unit.

The ventilation needle valves are configured to perform pressurization operations for the pressure-retaining cultivation cylinder and the pressure-retaining buffer cylinder, and the sampling needle valves are configured to perform pressure-retaining sampling operations for the pressure-retaining cultivation cylinder, the pressure-retaining buffer cylinder, and the monitoring compartment. The pressure sensors are configured to indicate the real-time pressures of the pressure-retaining cultivation cylinder, the pressure-retaining buffer cylinder, and the monitoring compartment.

The present invention further provides a multi-sequence integrity-preserving monitoring method for deep-sea microorganism cultivation, applied to the above apparatus and including:
S1: connecting, the gas pressurization unit, to all the pressure-retaining cultivation cylinders via the first multi-way valve, to the pressure-retaining buffer cylinder via the three-way valve, and to the monitoring compartment via the three-way valve and the four-way valve, and performing nitrogen pressurization to maintain all the pressure-retaining cultivation cylinders, the pressure-retaining buffer cylinder, and the monitoring compartment at a first pressure value;
S2: selecting a pressure-retaining cultivation cylinder to be monitored, opening a corresponding output end of the first multi-way valve, and performing, by the gas pressurization unit, nitrogen pressurization on the pressure-retaining cultivation cylinder to be monitored to increase a pressure of the first gas pressurization chamber to a second pressure value;
S3: opening a corresponding input end and a corresponding output end of the second multi-way valve, and the first port and the third port of the four-way valve, to enable communication between the pressure-retaining cultivation cylinder and the pressure-retaining buffer cylinder; and under a pressure difference between the second pressure value and the first pressure value, pushing, by the first piston in the pressure-retaining cultivation cylinder, a cultured sample in the pressure-retaining cultivation chamber to be injected into the pressure-retaining buffer chamber of the pressure-retaining buffer cylinder;
S4: closing the corresponding input end and the corresponding output end of the second multi-way valve, and the first port of the four-way valve, opening the first port and the third port of the three-way valve, and performing, by the gas pressurization unit, nitrogen pressurization on the pressure-retaining buffer cylinder to increase a pressure of the second gas pressurization chamber to the second pressure value;
S5: opening the second port of the four-way valve to enable communication between the pressure-retaining buffer cylinder and the monitoring compartment; and under the pressure difference between the second pressure value and the first pressure value, injecting, by the second piston in the pressure-retaining buffer cylinder, the cultured sample in the pressure-retaining buffer chamber into the compartment body of the monitoring compartment;
S6: when the cultured sample reaches the liquid level sensor in the monitoring compartment, closing the second port and the third port of the four-way valve; and monitoring the cultured sample, by the Raman spectroscopy probe, the ultraviolet spectroscopy probe, the compartment pressure sensor, and the water quality sensor, and transmitting obtained monitoring results to the sampling control unit;
S7: performing, by the gas pressurization unit, pressure reduction on the pressure-retaining buffer cylinder to decrease the pressure of the second gas pressurization chamber to a third pressure value;
S8: opening the second port and the third port of the four-way valve to enable

communication between the pressure-retaining buffer cylinder and the monitoring compartment; and under a pressure difference between the first pressure value and the third pressure value, sucking, by the second piston in the pressure-retaining buffer cylinder, the cultured sample in the monitoring compartment back to the pressure-retaining buffer chamber, and closing the second port and the third port of the four-way valve;
S9: performing, by the gas pressurization unit, nitrogen pressurization on the pressure-retaining buffer cylinder to increase the pressure of the second gas pressurization chamber to the second pressure value; and performing, by the gas pressurization unit, pressure reduction on the pressure-retaining cultivation cylinder to be monitored to decrease the pressure of the first gas pressurization chamber to the first pressure value;
S10: opening the corresponding input end and the corresponding output end of the second multi-way valve, and the first port and the third port of the four-way valve, to enable communication between the pressure-retaining cultivation cylinder and the pressure-retaining buffer cylinder; and under the pressure difference between the second pressure value and the first pressure value, pushing, by the second piston in the pressure-retaining buffer cylinder, the cultured sample in the pressure-retaining buffer chamber to be injected into the pressure-retaining cultivation chamber of the pressure-retaining cultivation cylinder;
S11: closing the corresponding input end and the corresponding output end of the second multi-way valve, and the first port and the third port of the four-way valve, to isolate the pressure-retaining cultivation cylinder, thus completing single-sequence integrity-preserving monitoring; and
S12: updating the selected pressure-retaining cultivation cylinder to be monitored, and repeating steps S2 to S11 to achieve multi-sequence integrity-preserving monitoring.

Preferably, the Raman spectroscopy probe monitors the cultured sample to obtain a sulfate concentration, a hydrogen sulfide concentration, and a formic acid concentration of the cultured sample.

The ultraviolet spectroscopy probe monitors the cultured sample to obtain an OD600, a nitrate concentration, a dissolved organic matter, and a total organic carbon concentration of the cultured sample.

The water quality sensor monitors the cultured sample to obtain one or more of a methane

concentration, a carbon dioxide concentration, a dissolved oxygen concentration, a pH value, a conductivity, and a temperature of the cultured sample.

Preferably, after completing a single integrity-preserving monitoring, the integrity-preserving monitoring unit is disassembled and cleaned, and after reconnection, the corresponding input end of the second multi-way valve is switched on to perform sampling and integrity-preserving monitoring of the remaining pressure-retaining cultivation cylinders.

As compared with the prior art, the technical solution of the present invention has the following beneficial effects:

In the present invention, the gas pressurization unit is configured to provide high-pressure nitrogen gas to the pressure-retaining cultivation unit and the integrity-preserving monitoring unit, achieving a high-pressure cultivation environment for deep-sea cultured samples. The gas pressurization unit achieves pressure-retaining transfer of deep-sea cultured samples between the pressure-retaining cultivation cylinder, the pressure-retaining buffer cylinder, and the monitoring compartment by changing the pressure values of the nitrogen gas injected into the pressure-retaining cultivation cylinder and the pressure-retaining buffer cylinder. The opening and closing of the second multi-way valve enable switching and sampling operations of multi-sequence pressure-retaining cultivation cylinders, and the single integrity-preserving monitoring unit is used to meet the demand for sampling and monitoring of multiple pressure-retaining cultivation cylinders, reducing equipment costs. The opening and closing of the three-way valve and the four-way valve achieve full-process pressure-retaining operations for sampling and recovery of cultured samples, ensuring the authenticity of cultured samples during monitoring, reducing losses of cultured samples during monitoring, improving the sampling frequency and efficiency of the cultivation process, and extending the cultivation cycle of deep-sea microorganisms.

### BRIEF DESCRIPTION OF THE DRAWINGS

FIG. 1 is a schematic structural diagram of a multi-sequence integrity-preserving monitoring apparatus for deep-sea microorganism cultivation according to Embodiment 1.

FIG. 2 is a schematic structural diagram of a multi-sequence integrity-preserving monitoring apparatus for deep-sea microorganism cultivation according to Embodiment 2.

FIG. 3 is a schematic structural diagram of a pressure-retaining cultivation cylinder according to Embodiment 2.

FIG. 4 is a schematic structural diagram of a pressure-retaining buffer cylinder according to Embodiment 2.

FIG. 5 is a schematic structural diagram of a monitoring compartment according to Embodiment 2.

FIG. 6 is a flowchart of a multi-sequence integrity-preserving monitoring method for deep-sea microorganism cultivation according to Embodiment 3.

### DESCRIPTION OF THE EMBODIMENTS

The drawings are for illustrative purposes only and should not be construed as limiting this patent.

To better illustrate embodiments, certain components in the drawings may be omitted, enlarged, or reduced, and do not represent the actual product dimensions.

For those skilled in the art, it is understandable that certain well-known structures in the drawings and their descriptions may be omitted.

The technical solution of the present invention is further described below with reference to the drawings and embodiments.

### Embodiment 1

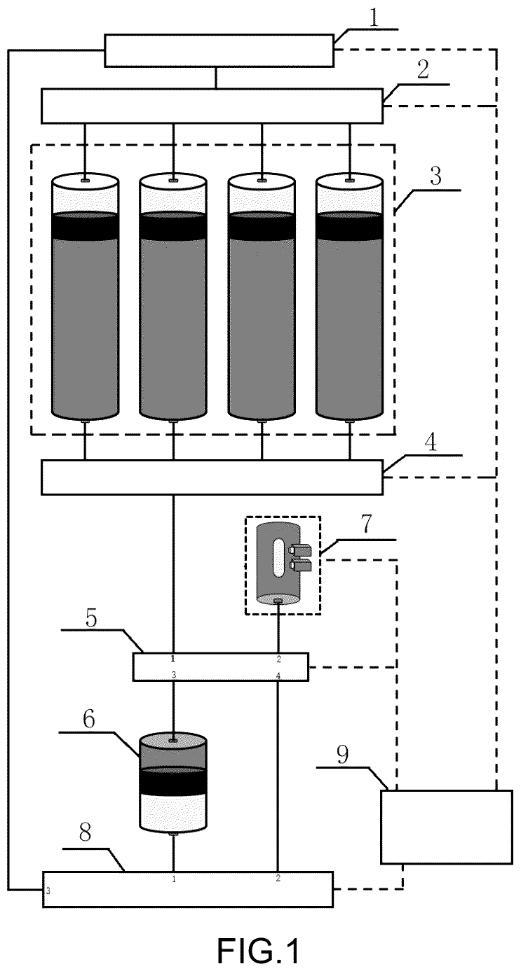

This embodiment provides a multi-sequence integrity-preserving monitoring apparatus for deep-sea microorganism cultivation, as shown in FIG. 1, including a gas pressurization unit 1, a pressure-retaining cultivation unit, an integrity-preserving monitoring unit, and a sampling control unit 9.

The pressure-retaining cultivation unit includes a first multi-way valve 2, a plurality of pressure-retaining cultivation cylinders 3, and a second multi-way valve 4, where an input end of the first multi-way valve 2 is connected to a first output end of the gas pressurization unit 1, a plurality of output ends of the first multi-way valve 2 are each connected to one end of one pressure-retaining cultivation cylinder 3 correspondingly, and the other end of each of the pressure-retaining cultivation cylinders 3 is connected to one input end of the second multi-way valve 4 correspondingly.

The integrity-preserving monitoring unit includes a three-way valve 8, a four-way valve 5, a pressure-retaining buffer cylinder 6, and a monitoring compartment 7, where a first port of the four-way valve 5 is connected to an output end of the second multi-way valve 4, and a second port of the four-way valve 5 is connected to an input end of the monitoring compartment 7; a third port of the four-way valve 5 is connected to one end of the pressure-retaining buffer cylinder 6, and the other end of the pressure-retaining buffer cylinder 6 is connected to a first port of the three-way valve 8; and a fourth port of the four-way valve 5 is connected to a second port of the three-way valve 8, and a third port of the three-way valve 8 is connected to a second output end of the gas pressurization unit 1.

An input end of the sampling control unit 9 is connected to a data output end of the monitoring compartment 7, and an output end of the sampling control unit 9 is connected to a control end of the gas pressurization unit 1, a control end of the first multi-way valve 2, a control end of the second multi-way valve 4, a control end of the three-way valve 8, and a control end of the four-way valve 5.

In a specific implementation process, the gas pressurization unit 1 is configured to provide high-pressure nitrogen gas to the pressure-retaining cultivation unit and the integrity-preserving monitoring unit, achieving a high-pressure cultivation environment for deep-sea cultured samples. The gas pressurization unit 1 achieves transfer of deep-sea cultured samples between the pressure-retaining cultivation cylinder 3, the pressure-retaining buffer cylinder 6, and the monitoring compartment 7 by changing the pressure values of nitrogen gas injected into the pressure-retaining cultivation cylinder 3 and the pressure-retaining buffer cylinder 6. The opening and closing of the second multi-way valve 4 enable switching and sampling operations of multiple pressure-retaining cultivation cylinders 3, and the single integrity-preserving monitoring unit is used to meet the demand for sampling and monitoring of multiple pressure-retaining cultivation cylinders 3, reducing equipment costs. The opening and closing of the three-way valve 8 and the four-way valve 5 achieve full-process pressure-retaining operations for sampling and recovery of cultured samples, ensuring the authenticity of cultured samples during monitoring, reducing losses of cultured samples during monitoring, improving the sampling frequency and efficiency of the cultivation process, and extending the cultivation cycle of deep-sea microorganisms.

### Embodiment 2

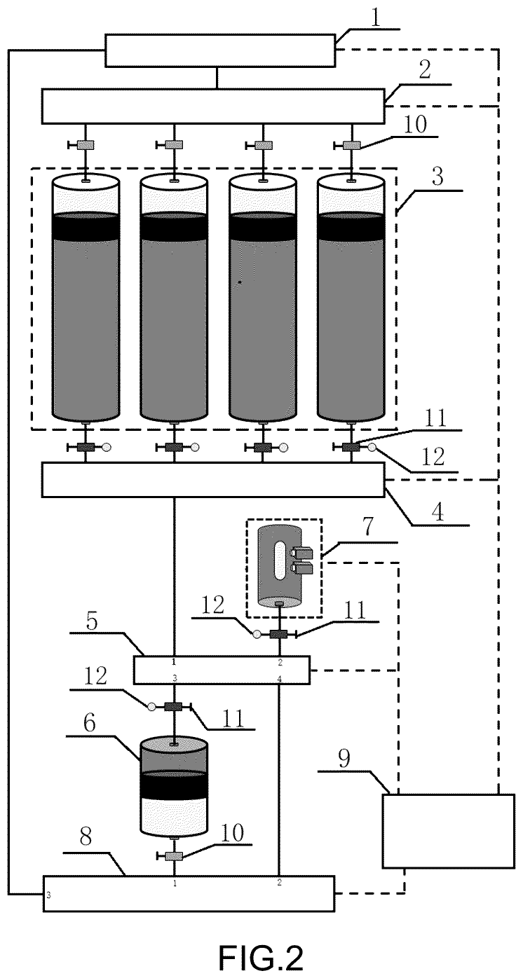

This embodiment provides a multi-sequence integrity-preserving monitoring apparatus for deep-sea microorganism cultivation, as shown in FIG. 2, including a gas pressurization unit 1, a pressure-retaining cultivation unit, an integrity-preserving monitoring unit, and a sampling control unit 9, a plurality of sampling needle valves 10, a plurality of ventilation needle valves 11, and a plurality of pressure sensors 12.

The pressure-retaining cultivation unit includes a first multi-way valve 2, a plurality of pressure-retaining cultivation cylinders 3, and a second multi-way valve 4, where an input end of the first multi-way valve 2 is connected to a first output end of the gas pressurization unit 1, a plurality of output ends of the first multi-way valve 2 are each connected to one end of one pressure-retaining cultivation cylinder 3 correspondingly, and the other end of each of the pressure-retaining cultivation cylinders 3 is connected to one input end of the second multi-way valve 4 correspondingly; and one ventilation needle valve 11 is disposed between each output end of the first multi-way valve 2 and one end of one pressure-retaining cultivation cylinder 3.

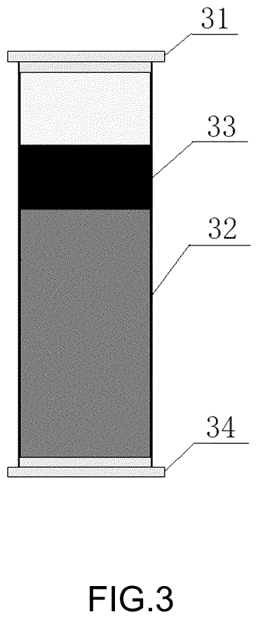

As shown in FIG. 3, each of the pressure-retaining cultivation cylinders 3 includes a gas sealing end cap 31, a first cavity 32, a first piston 33, and a seawater sealing end cap 34.

The first piston 33 is disposed inside the first cavity 32 and divides the first cavity 32 into a first gas pressurization chamber and a pressure-retaining cultivation chamber; and the gas sealing end cap 31 is disposed at one end of the first gas pressurization chamber, and the seawater sealing end cap 34 is disposed at one end of the pressure-retaining cultivation chamber.

The plurality of output ends of the first multi-way valve 2 are each connected to the gas sealing end cap 31 of one pressure-retaining cultivation cylinder 3 correspondingly, and the seawater sealing end cap 34 of each of the pressure-retaining cultivation cylinders 3 is connected to one input end of the second multi-way valve 4 correspondingly.

The first cavity 32 is a titanium alloy cylindrical structure with a built-in movable first piston 33, and the first piston 33 is fitted with an O-ring seal, seperating the first cavity 32 into the first gas pressurization chamber and the pressure-retaining cultivation chamber. The gas pressurization unit 1 injects high-pressure nitrogen gas into the first gas pressurization chamber of the pressure-retaining cultivation cylinder 3 via the first multi-way valve 2, achieving a high-pressure cultivation environment for deep-sea cultured samples.

The integrity-preserving monitoring unit includes a three-way valve 8, a four-way valve 5, a pressure-retaining buffer cylinder 6, and a monitoring compartment 7, where a first port of the four-way valve 5 is connected to an output end of the second multi-way valve 4, and a second port of the four-way valve 5 is connected to an input end of the monitoring compartment 7; a third port of the four-way valve 5 is connected to one end of the pressure-retaining buffer cylinder 6, and the other end of the pressure-retaining buffer cylinder 6 is connected to a first port of the three-way valve 8; and a fourth port of the four-way valve 5 is connected to a second port of the three-way valve 8, and a third port of the three-way valve 8 is connected to a second output end of the gas pressurization unit 1. One ventilation needle valve 11 is disposed between the other end of the pressure-retaining buffer cylinder 6 and the first port of the three-way valve 8; one sampling needle valve 10 is disposed between the other end of each of the pressure-retaining cultivation cylinders 3 and one input end of the second multi-way valve 4; one sampling needle valve 10 is disposed between the third port of the four-way valve 5 and one end of the pressure-retaining buffer cylinder 6; and one sampling needle valve 10 is disposed between the second port of the four-way valve 5 and the input end of the monitoring compartment 7. Each of the pressure sensors 12 is disposed at a sampling end of one sampling needle valve 10 correspondingly.

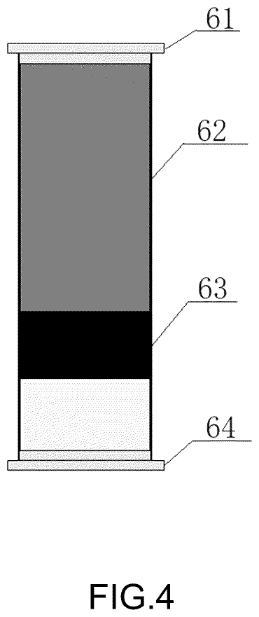

As shown in FIG. 4, the pressure-retaining buffer cylinder 6 includes a first sealing end cap 61, a second cavity 62, a second piston 63, and a second sealing end cap 64.

The second piston 63 is disposed inside the second cavity 62 and divides the second cavity 62 into a second gas pressurization chamber and a pressure-retaining buffer chamber; and the first sealing end cap 61 is disposed at one end of the pressure-retaining buffer chamber, and the second sealing end cap 64 is disposed at one end of the second gas pressurization chamber.

The third port of the four-way valve 5 is connected to the first sealing end cap 61 of the pressure-retaining buffer cylinder 6, and the second sealing end cap 64 of the pressure-retaining buffer cylinder 6 is connected to the first port of the three-way valve 8.

The second cavity 62 is a titanium alloy cylindrical structure with a built-in movable second piston 63, and the second piston 63 is fitted with an O-ring seal, dividing the second cavity 62 into the second gas pressurization chamber and the pressure-retaining buffer chamber. The gas pressurization unit 1 injects high-pressure nitrogen gas into the second gas pressurization chamber of the pressure-retaining buffer cylinder 6 via the three-way valve 8, achieving a high-pressure cultivation environment for deep-sea cultured samples. The cultured sample in the pressure-retaining cultivation cylinder 3 enters the pressure-retaining buffer chamber of the pressure-retaining buffer cylinder 3 via the second multi-way valve 4 and the four-way valve 5, achieving pressure-retaining buffering of the cultured sample.

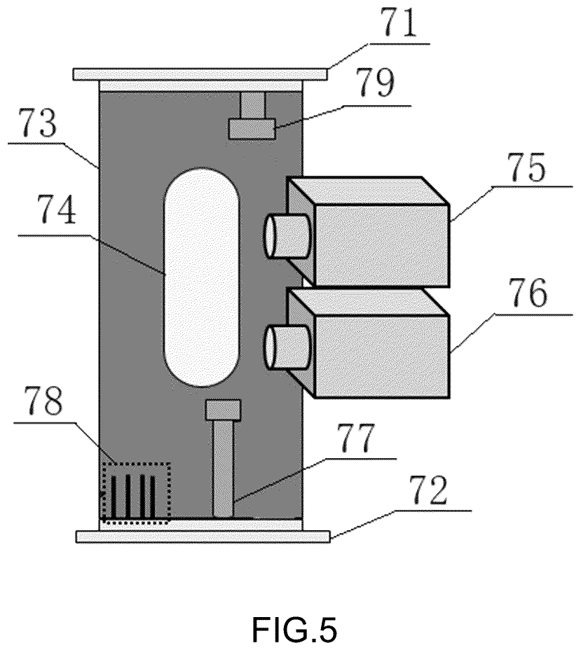

As shown in FIG. 5, the monitoring compartment 7 includes a third sealing end cap 71, a fourth sealing end cap 72, a compartment body 73, a viewing port 74, a Raman spectroscopy probe 75, an ultraviolet spectroscopy probe 76, a compartment pressure sensor 77, a water quality sensor 78, and a liquid level sensor 79.

The third sealing end cap 71 and the fourth sealing end cap 72 are respectively disposed at two ends of the compartment body 73, and the viewing port 74 is disposed on a sidewall of the compartment body 73; and the liquid level sensor 79 is disposed on a lower surface of the third sealing end cap 71, the water quality sensor 78 and the compartment pressure sensor 77 are disposed on an upper surface of the fourth sealing end cap 72, and both the Raman spectroscopy probe 75 and the ultraviolet spectroscopy probe 76 are disposed on the viewing port 74.

The second port of the four-way valve 5 is connected to a lower surface of the fourth sealing end cap 72 of the monitoring compartment 7.

Data output ends of the Raman spectroscopy probe 75, the ultraviolet spectroscopy probe 76, the water quality sensor 78, and the liquid level sensor 79 are all connected to the input end of the sampling control unit 9.

The compartment body 73 is a titanium alloy cylindrical structure, and the fourth sealing end cap 72 is communicated with the four-way valve 5 for injection and outflow of cultured samples. The compartment pressure sensor 77 and the liquid level sensor 79 are configured to monitor the pressure value and liquid level height of the cultured sample in the compartment body 73 in real time, reflecting the sample volume in the compartment body 73. The Raman spectroscopy probe 75, the ultraviolet spectroscopy probe 76, and the water quality sensor 78 monitor multiple physicochemical parameters of the cultured sample in the compartment body 73 in real time. The gas pressurization unit 1 injects high-pressure nitrogen gas into the compartment body 73 via the three-way valve 8 and the four-way valve 5, achieving a high-pressure monitoring environment for deep-sea cultured samples.

The water quality sensor 78 includes any one or more of a methane sensor, a carbon dioxide sensor, a dissolved oxygen sensor, a temperature sensor, a conductivity sensor, and a pH sensor. The dissolved oxygen sensor, the temperature sensor, the conductivity sensor, and the pH sensor are all probe-type sensors.

Data output ends of each of the pressure sensors 12, the Raman spectroscopy probe 75, the ultraviolet spectroscopy probe 76, the water quality sensor 78, and the liquid level sensor 79 are all connected to the input end of the sampling control unit 9, and an output end of the sampling control unit 9 is connected to a control end of the gas pressurization unit 1, a control end of the first multi-way valve 2, a control end of the second multi-way valve 4, a control end of the three-way valve 8, and a control end of the four-way valve 5.

### Embodiment 3

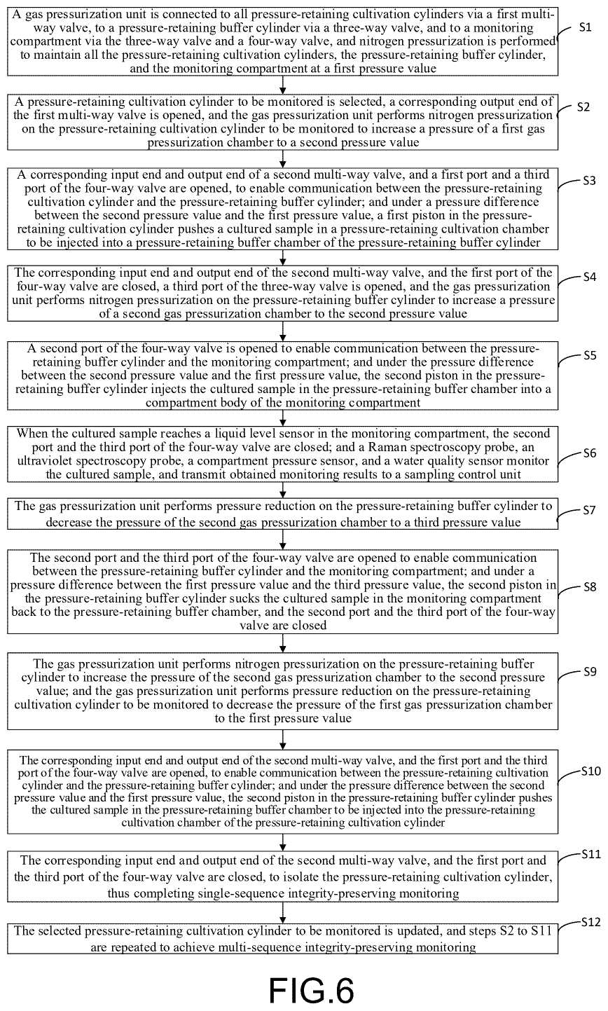

This embodiment provides a multi-sequence integrity-preserving monitoring method for deep-sea microorganism cultivation, applied to the apparatus described in Embodiment 1 or 2, as shown in FIG. 6, and including:
S1: connecting, the gas pressurization unit, to all the pressure-retaining cultivation cylinders via the first multi-way valve, to the pressure-retaining buffer cylinder via the three-way valve, and to the monitoring compartment via the three-way valve and the four-way valve, and performing nitrogen pressurization to maintain all the pressure-retaining cultivation cylinders, the pressure-retaining buffer cylinder, and the monitoring compartment at a first pressure value;
S2: selecting a pressure-retaining cultivation cylinder to be monitored, opening a corresponding output end of the first multi-way valve, and performing, by the gas pressurization unit, nitrogen pressurization on the pressure-retaining cultivation cylinder to be monitored to increase a pressure of the first gas pressurization chamber to a second pressure value;
S3: opening a corresponding input end and a corresponding output end of the second multi-way valve, and the first port and the third port of the four-way valve, to enable communication between the pressure-retaining cultivation cylinder and the pressure-retaining buffer cylinder; and under a pressure difference between the second pressure value and the first pressure value, pushing, by the first piston in the pressure-retaining cultivation cylinder, a cultured sample in the pressure-retaining cultivation chamber to be injected into the pressure-retaining buffer chamber of the pressure-retaining buffer cylinder;
S4: closing the corresponding input end and the corresponding output end of the second multi-way valve, and the first port of the four-way valve, opening the first port and the third port of the three-way valve, and performing, by the gas pressurization unit, nitrogen pressurization on the pressure-retaining buffer cylinder to increase a pressure of the second gas pressurization chamber to the second pressure value;
S5: opening the second port of the four-way valve to enable communication between the pressure-retaining buffer cylinder and the monitoring compartment; and under the pressure difference between the second pressure value and the first pressure value, injecting, by the second piston in the pressure-retaining buffer cylinder, the cultured sample in the pressure-retaining buffer chamber into the compartment body of the monitoring compartment;
S6: when the cultured sample reaches the liquid level sensor in the monitoring compartment, closing the second port and the third port of the four-way valve; and monitoring the cultured sample, by the Raman spectroscopy probe, the ultraviolet spectroscopy probe, the compartment pressure sensor, and the water quality sensor, and transmitting obtained monitoring results to the sampling control unit;
S7: performing, by the gas pressurization unit, pressure reduction on the pressure-retaining buffer cylinder to decrease the pressure of the second gas pressurization chamber to a third pressure value;
S8: opening the second port and the third port of the four-way valve to enable communication between the pressure-retaining buffer cylinder and the monitoring compartment; and under a pressure difference between the first pressure value and the third pressure value, sucking, by the second piston in the pressure-retaining buffer cylinder, the cultured sample in the monitoring compartment back to the pressure-retaining buffer chamber, and closing the second port and the third port of the four-way valve;
S9: performing, by the gas pressurization unit, nitrogen pressurization on the pressure-retaining buffer cylinder to increase the pressure of the second gas pressurization chamber to the second pressure value; and performing, by the gas pressurization unit, pressure reduction on the pressure-retaining cultivation cylinder to be monitored to decrease the pressure of the first gas pressurization chamber to the first pressure value;
S10: opening the corresponding input end and the corresponding output end of the second multi-way valve, and the first port and the third port of the four-way valve, to enable communication between the pressure-retaining cultivation cylinder and the pressure-retaining buffer cylinder; and under the pressure difference between the second pressure value and the first pressure value, pushing, by the second piston in the pressure-retaining buffer cylinder, the cultured sample in the pressure-retaining buffer chamber to be injected into the pressure-retaining cultivation chamber of the pressure-retaining cultivation cylinder;
S11: closing the corresponding input end and output end of the second multi-way valve, and the first port and the third port of the four-way valve, to isolate the pressure-retaining cultivation cylinder, thus completing single-sequence integrity-preserving monitoring; and
S12: updating the selected pressure-retaining cultivation cylinder to be monitored, and repeating steps S2 to S11 to achieve multi-sequence integrity-preserving monitoring.

In a specific implementation process, during the long-cycle pressure-retaining cultivation of deep-sea cultured samples, the pressure values of the pressure sensors in the pressure-retaining cultivation unit are periodically checked, and the pressure-retaining cultivation is connected to the gas pressurization unit via the first multi-way valve to ensure that the pressure-retaining environment of the multi-sequence cultivation system is maintained at the in-situ deep-sea pressure value, that is, the first pressure value. During the integrity-preserving monitoring process, the integrity-preserving monitoring unit is connected to the gas pressurization unit via the three-way valve and the four-way valve, and through nitrogen pressurization, the pressure values of the monitoring compartment and the second gas pressurization chamber of the pressure-retaining buffer cylinder are also raised to the first pressure value, resulting in no pressure difference between the pressure-retaining cultivation unit and the integrity-preserving monitoring unit.

A pressure-retaining cultivation cylinder to be monitored is selected, a corresponding output end of the first multi-way valve is opened, and the gas pressurization unit performs nitrogen pressurization on the pressure-retaining cultivation cylinder to be monitored to increase the pressure of the first gas pressurization chamber to a second pressure value. Simultaneously, a corresponding input end and a corresponding output end of the second multi-way valve, and the first port and the third port of the four-way valve are opened to enable communication between the pressure-retaining cultivation cylinder and the pressure-retaining buffer cylinder. Due to the pressure value of the pressure-retaining cultivation chamber being higher than the pressure value of the pressure-retaining buffer chamber, under the pressure difference, the first piston pushes the cultured sample in the pressure-retaining cultivation chamber to be injected into the pressure-retaining buffer chamber.

After the cultured sample transfer is completed, the corresponding input end and the corresponding output end of the second multi-way valve and the first port of the four-way valve are closed. The gas pressurization unit performs nitrogen pressurization on the pressure-retaining buffer cylinder to increase the pressure of the second gas pressurization chamber to the second pressure value. The second port of the four-way valve is opened to enable communication between the pressure-retaining buffer cylinder and the monitoring compartment. Due to the pressure value of the pressure-retaining buffer chamber being higher than the pressure value of the monitoring compartment, under the pressure difference, the second piston pushes the cultured sample in the pressure-retaining buffer chamber to be injected into the monitoring compartment. The liquid level sensor is used to determine whether the cultured sample has filled the monitoring compartment, and upon filling, the second port and the third port of the four-way valve are closed to stop injection. The material of the viewing port of the monitoring compartment is sapphire. The Raman spectroscopy probe outside the viewing port performs monitoring to obtain a sulfate concentration, a hydrogen sulfide concentration, and a formic acid concentration of the cultured sample. The ultraviolet spectroscopy probe performs monitoring to obtain an OD600, a nitrate concentration, a dissolved organic matter, and a total organic carbon concentration of the cultured sample. The water quality sensor performs monitoring to obtain a methane concentration, a carbon dioxide concentration, a dissolved oxygen concentration, a pH value, a conductivity, and a temperature of the cultured sample. Data output ends of the water quality sensor, the Raman spectroscopy probe, and the ultraviolet spectroscopy probe are connected to the sampling control unit, transmitting the monitoring results to the sampling control unit for solving.

After the integrity-preserving monitoring operation is completed, the cultured sample in the monitoring compartment needs to be recovered back to the pressure-retaining cultivation cylinder. The gas pressurization unit performs pressure reduction on the pressure-retaining buffer cylinder to decrease the pressure of the second gas pressurization chamber to a third pressure value, and the second port and the third port of the four-way valve are opened to enable communication between the pressure-retaining buffer cylinder and the monitoring compartment. Due to the pressure value of the monitoring compartment being higher than the pressure value of the pressure-retaining buffer chamber, under the pressure difference, the second piston moves to suck the cultured sample in the monitoring compartment back to the pressure-retaining buffer chamber, and the second port and the third port of the four-way valve are closed.

The gas pressurization unit performs nitrogen pressurization on the pressure-retaining buffer cylinder to increase the pressure of the second gas pressurization chamber to the second pressure value, while performing pressure reduction on the pressure-retaining cultivation cylinder to be monitored to decrease the pressure of the first gas pressurization chamber to the first pressure value. The corresponding input end and the corresponding output end of the second multi-way valve, and the first port and the third port of the four-way valve are opened to enable communication between the pressure-retaining cultivation cylinder and the pressure-retaining buffer cylinder. Due to the pressure value of the pressure-retaining buffer chamber being higher than the pressure value of the pressure-retaining cultivation chamber, the second piston in the pressure-retaining buffer cylinder pushes the cultured sample in the pressure-retaining buffer chamber to be injected into the pressure-retaining cultivation chamber of the pressure-retaining cultivation cylinder. The corresponding input end and the corresponding output end of the second multi-way valve, and the first port and the third port of the four-way valve are closed to isolate the pressure-retaining cultivation cylinder, thus completing single integrity-preserving monitoring.

Based on the above sample recovery process, the addition of culture medium or nutrients to the pressure-retaining buffer chamber enables replenishment operations for the pressure-retaining cultivation unit. After a single integrity-preserving monitoring is completed, the integrity-preserving monitoring unit is disassembled and cleaned. After reconnection, the corresponding input end of the second multi-way valve is switched on to perform sampling and integrity-preserving monitoring of the remaining pressure-retaining cultivation cylinders.

The method provided in this embodiment enables enrichment cultivation, low-loss integrity-preserving sampling, and monitoring of deep-sea microorganisms, improving the authenticity of cultured sample monitoring. The low-loss approach allows for increased sampling frequency of cultured samples, extending the time cycle of the deep-sea microorganism enrichment cultivation process.

Identical or similar reference numerals correspond to identical or similar components.

Terms describing positional relationships in the drawings are for illustrative purposes only and should not be construed as limiting this patent.

Obviously, the above embodiments of the present invention are merely examples to clearly illustrate the present invention and are not intended to limit the implementations of the present invention. For those of ordinary skill in the art, other variations or modifications in different forms can be made based on the above description. It is neither necessary nor possible to exhaustively list all implementations herein. Any modifications, equivalent substitutions, improvements, and the like made within the spirit and principles of the present invention shall be included within the scope of protection of the claims of the present invention.

What is claimed is:

1. A multi-sequence integrity-preserving monitoring apparatus for deep-sea microorganism cultivation, comprising a gas pressurization unit, a pressure-retaining cultivation unit, an integrity-preserving monitoring unit, and a sampling control unit; wherein
the pressure-retaining cultivation unit comprises a first multi-way valve, a plurality of pressure-retaining cultivation cylinders, and a second multi-way valve, wherein an input end of the first multi-way valve is connected to a first output end of the gas pressurization unit, a plurality of output ends of the first multi-way valve are each connected to one end of one pressure-retaining cultivation cylinder correspondingly, and the other end of each of the pressure-retaining cultivation cylinders is connected to one input end of the second multi-way valve correspondingly;
the integrity-preserving monitoring unit comprises a four-way valve, a three-way valve, a pressure-retaining buffer cylinder, and a monitoring compartment, wherein a first port of the four-way valve is connected to an output end of the second multi-way valve, and a second port of the four-way valve is connected to an input end of the monitoring compartment; a third port of the four-way valve is connected to one end of the pressure-retaining buffer cylinder, and the other end of the pressure-retaining buffer cylinder is connected to a first port of the three-way valve; and a fourth port of the four-way valve is connected to a second port of the three-way valve, and a third port of the three-way valve is connected to a second output end of the gas pressurization unit;
an input end of the sampling control unit is connected to a data output end of the monitoring compartment, and an output end of the sampling control unit is connected to a control end of the gas pressurization unit, a control end of the first multi-way valve, a control end of the second multi-way valve, a control end of the three-way valve, and a control end of the four-way valve;
each of the pressure-retaining cultivation cylinders comprises a gas sealing end cap, a first cavity, a first piston, and a seawater sealing end cap;
the first piston is disposed inside the first cavity and divides the first cavity into a first gas pressurization chamber and a pressure-retaining cultivation chamber; and the gas sealing end cap is disposed at one end of the first gas pressurization chamber, and the seawater sealing end cap is disposed at one end of the pressure-retaining cultivation chamber;
the plurality of output ends of the first multi-way valve are each connected to the gas sealing end cap of one pressure-retaining cultivation cylinder correspondingly, and the seawater sealing end cap of each of the pressure-retaining cultivation cylinders is connected to one input end of the second multi-way valve correspondingly;
the pressure-retaining buffer cylinder comprises a first sealing end cap, a second cavity, a second piston, and a second sealing end cap;
the second piston is disposed inside the second cavity and divides the second cavity into a second gas pressurization chamber and a pressure-retaining buffer chamber; and the first sealing end cap is disposed at one end of the pressure-retaining buffer chamber, and the second sealing end cap is disposed at one end of the second gas pressurization chamber; and
the third port of the four-way valve is connected to the first sealing end cap of the pressure-retaining buffer cylinder, and the second sealing end cap of the pressure-retaining buffer cylinder is connected to the first port of the three-way valve.

2. The multi-sequence integrity-preserving monitoring apparatus for deep-sea microorganism cultivation according to claim 1, wherein the monitoring compartment comprises a third sealing end cap, a fourth sealing end cap, a compartment body, a viewing port, a Raman spectroscopy probe, an ultraviolet spectroscopy probe, a compartment pressure sensor, a water quality sensor, and a liquid level sensor; wherein
the third sealing end cap and the fourth sealing end cap are respectively disposed at two ends of the compartment body, and the viewing port is disposed on a sidewall of the compartment body; and the liquid level sensor is disposed on a lower surface of the third sealing end cap, the water quality sensor and the compartment pressure sensor are disposed on an upper surface of the fourth sealing end cap, and the Raman spectroscopy probe and the ultraviolet spectroscopy probe are all disposed on the viewing port;
the second port of the four-way valve is connected to a lower surface of the fourth sealing end cap of the monitoring compartment; and
data output ends of the Raman spectroscopy probe, the ultraviolet spectroscopy probe, the water quality sensor, and the liquid level sensor are all connected to the input end of the sampling control unit.

3. The multi-sequence integrity-preserving monitoring apparatus for deep-sea microorganism cultivation according to claim 2, wherein the water quality sensor comprises any one or more of a methane sensor, a carbon dioxide sensor, a dissolved oxygen sensor, a temperature sensor, a conductivity sensor, and a pH sensor.

4. The multi-sequence integrity-preserving monitoring apparatus for deep-sea microorganism cultivation according to claim 3, wherein the dissolved oxygen sensor, the temperature sensor, the conductivity sensor, and the pH sensor are all probe-type sensors.

5. The multi-sequence integrity-preserving monitoring apparatus for deep-sea microorganism cultivation according to claim 1, wherein the multi-sequence integrity-preserving monitoring apparatus further comprises a plurality of sampling needle valves and a plurality of ventilation needle valves; wherein
one ventilation needle valve is disposed between each of the output ends of the first multi-way valve and one end of one pressure-retaining cultivation cylinder;
one ventilation needle valve is disposed between the other end of the pressure-retaining buffer cylinder and the first port of the three-way valve;
one sampling needle valve is disposed between the other end of each of the pressure-retaining cultivation cylinders and one input end of the second multi-way valve;
one sampling needle valve is disposed between the third port of the four-way valve and one end of the pressure-retaining buffer cylinder; and
one sampling needle valve is disposed between the second port of the four-way valve and the input end of the monitoring compartment.

6. The multi-sequence integrity-preserving monitoring apparatus for deep-sea microorganism cultivation according to claim 1, wherein the multi-sequence integrity-preserving monitoring apparatus further comprises a plurality of pressure sensors; wherein
each of the pressure sensors is disposed at a sampling end of one sampling needle valve correspondingly, and a data output end of each of the pressure sensors is connected to the input end of the sampling control unit.

7. A multi-sequence integrity-preserving monitoring method for deep-sea microorganism cultivation, applied to the multi-sequence integrity-preserving monitoring apparatus according to claim 2, and comprising:
S1: connecting, the gas pressurization unit, to all the pressure-retaining cultivation cylinders via the first multi-way valve, to the pressure-retaining buffer cylinder via the three-way valve, and to the monitoring compartment via the three-way valve and the four-way valve, and performing nitrogen pressurization to maintain all the pressure-retaining cultivation cylinders, the pressure-retaining buffer cylinder, and the monitoring compartment at a first pressure value;
S2: selecting a pressure-retaining cultivation cylinder to be monitored, opening a corresponding output end of the first multi-way valve, and performing, by the gas pressurization unit, nitrogen pressurization on the pressure-retaining cultivation cylinder to be monitored to increase a pressure of the first gas pressurization chamber to a second pressure value;
S3: opening a corresponding input end and a corresponding output end of the second multi-way valve, and the first port and the third port of the four-way valve, to enable communication between the pressure-retaining cultivation cylinder and the pressure-retaining buffer cylinder; and
under a pressure difference between the second pressure value and the first pressure value, pushing, by the first piston in the pressure-retaining cultivation cylinder, a cultured sample in the pressure-retaining cultivation chamber to be injected into the pressure-retaining buffer chamber of the pressure-retaining buffer cylinder;
S4: closing the corresponding input end and the corresponding output end of the second multi-way valve, and the first port of the four-way valve, opening the first port and the third port of the three-way valve, and performing, by the gas pressurization unit, nitrogen pressurization on the pressure-retaining buffer cylinder to increase a pressure of the second gas pressurization chamber to the second pressure value;
S5: opening the second port of the four-way valve to enable communication between the pressure-retaining buffer cylinder and the monitoring compartment; and under the pressure difference between the second pressure value and the first pressure value, pushing, by the second piston in the pressure-retaining buffer cylinder, the cultured sample in the pressure-retaining buffer chamber to be injected into the compartment body of the monitoring compartment;
S6: when the cultured sample reaches the liquid level sensor in the monitoring compartment, closing the second port and the third port of the four-way valve; and monitoring the cultured sample, by the Raman spectroscopy probe, the ultraviolet spectroscopy probe, the compartment pressure sensor, and the water quality sensor, and transmitting obtained monitoring results to the sampling control unit;
S7: performing, by the gas pressurization unit, pressure reduction on the pressure-retaining buffer cylinder to decrease the pressure of the second gas pressurization chamber to a third pressure value;
S8: opening the second port and the third port of the four-way valve to enable communication between the pressure-retaining buffer cylinder and the monitoring compartment; and under a pressure difference between the first pressure value and the third pressure value, sucking, by the second piston in the pressure-retaining buffer cylinder, the cultured sample in the monitoring compartment back to the pressure-retaining buffer chamber, and closing the second port and the third port of the four-way valve;
S9: performing, by the gas pressurization unit, nitrogen pressurization on the pressure-retaining buffer cylinder to increase the pressure of the second gas pressurization chamber to the second pressure value; and performing, by the gas pressurization unit, pressure reduction on the pressure-retaining cultivation cylinder to be monitored to decrease the pressure of the first gas pressurization chamber to the first pressure value;
S10: opening the corresponding input end and the corresponding output end of the second multi-way valve, and the first port and the third port of the four-way valve, to enable communication between the pressure-retaining cultivation cylinder and the pressure-retaining buffer cylinder; and under the pressure difference between the second pressure value and the first pressure value, pushing, by the second piston in the pressure-retaining buffer cylinder, the cultured sample in the pressure-retaining buffer chamber to be injected into the pressure-retaining cultivation chamber of the pressure-retaining cultivation cylinder;
S11: closing the corresponding input end and the corresponding output end of the second multi-way valve, and the first port and the third port of the four-way valve, to isolate the pressure-retaining cultivation cylinder, thus completing single-sequence integrity-preserving monitoring; and
S12: updating the selected pressure-retaining cultivation cylinder to be monitored, and repeating steps S2 to S11 to achieve multi-sequence integrity-preserving monitoring.

8. The multi-sequence integrity-preserving monitoring method for deep-sea microorganism cultivation according to claim 7, wherein the Raman spectroscopy probe monitors the cultured sample to obtain a sulfate concentration, a hydrogen sulfide concentration, and a formic acid concentration of the cultured sample;
the ultraviolet spectroscopy probe monitors the cultured sample to obtain an OD600, a nitrate concentration. a dissolved organic matter, and a total organic carbon concentration of the cultured sample; and the water quality sensor monitors the cultured sample to obtain one or more of a methane concentration, a carbon dioxide concentration, a dissolved oxygen concentration, a pH value, a conductivity, and a temperature of the cultured sample.
